(19) United States
(12) Patent Application Publication
(10) Pub. No.: US 2025/0057800 A1
(43) Pub. Date: Feb. 20, 2025

(54) DRUG DELIVERY SYSTEM COMPRISING MONOMETHYL FUMARATE

(71) Applicant: GENCAPS SÀRL, Capellen (LU)

(72) Inventor: Sébastien GRELLETY, Nernier (FR)

(21) Appl. No.: 18/723,270

(22) PCT Filed: Dec. 7, 2022
(86) PCT No.: PCT/IB2022/061884

§ 371 Date: Jun. 24, 2024

(30) Foreign Application Priority Data
Dec. 23, 2021 (WO) PCT/IB2021/062244

(51) Int. Cl. A61K 31/22; A61K 9/48

(52) U.S. Cl. CPC A61K 31/22; A61K 9/4808; A61K 9/4858

(57) ABSTRACT

A sustained release drug delivery system comprising a monolithic solid polyethylene glycol (PEG) matrix and monomethyl fumarate (MMF) characterized by the following conditions: —the MMF being dispersed under solid state in the said matrix, and—the system comprising from 50% to 60% w/w of MMF. Said system for use in the treatment of an autoimmune disease wherein the autoimmune disease is preferably psoriasis or multiple sclerosis.

### FIELD OF THE INVENTION

The present invention relates to a drug delivery system comprising monomethyl fumarate and to the use of said drug delivery system as a pharmaceutical formulation.

### BACKGROUND

Dimethyl fumarate (DMF) is a known pharmaceutically active ingredient that can be used to treat a variety of diseases including the autoimmune diseases psoriasis and multiple sclerosis. However, whilst DMF can be therapeutically effective, its oral administration can cause side effects e.g. flushing and gastrointestinal complaints such as nausea and vomiting. These side effects can impact patient compliance, and in severe cases lead to discontinuation of treatment.

It is thought that certain side effects e.g. gastrointestinal side effects, of orally administered DMF can be reduced by minimising the concentration of DMF released at one time within the stomach and/or gastrointestinal tract. It has therefore been a focus of pharmaceutical companies to develop oral drug delivery systems that may achieve this aim.

One such oral drug delivery system is disclosed in WO2013/119677 which describes a capsule comprising multiple enterically coated mini-tablets comprising DMF. Said drug delivery system is designed to reduce or prevent the release of DMF in the stomach. However, high concentrations of DMF can still be released locally at one time in the gastrointestinal tract e.g., small intestine, which can still lead to side effects.

The active metabolite of DMF after oral administration is monomethyl fumarate (MMF). Indeed, DMF is not detectable in the systemic circulation, since it is rapidly hydrolyzed to MMF by esterases present in the gastrointestinal tract. It is well-known that when DMF is hydrolysed to MMF it produces methanol (a known gastrointestinal irritant) as a by-product in the small intestine lumen and mucosa. This localized methanol production within the gastrointestinal tract may contribute to DMF gastrointestinal tolerability issues (Michael J. Palte, Angela Wehr, Mark Tawa, Kristopher Perkin. Improving the gastrointestinal tolerability of FAE: early findings on gastrointestinal events with Diroximel Fumarate in patients with relapsing-remitting multiple sclerosis from the phase 3, open label EVOLVE-MS-1 study. Adv Ther (2019) 36:3154-3165).

Existing commercial products such as Tecfidera® consist of an oral formulation containing 240 mg of DMF, with an average in vitro release rate (paddle speed 40 rpm) in the range of about 5% of the dose per minute at 37° C. in 300 ml Fasted State Simulated Intestinal Fluid (FaSSIF) at pH 6.5. Thus, more than 90% of the 240 mg dose of DMF, i.e. 216 mg of DMF, is predicted to be released in less than 20 minutes in the gastrointestinal tract leading to high local concentrations of DMF.

MMF instead of DMF may be used to reduce or even avoid high local concentrations of DMF or its active metabolite in the gastrointestinal tract, and therefore may be able to reduce the side effects associated with the oral administration of DMF.

However, the release rate of MMF in the gastrointestinal tract still remains relatively important, in such a way that the local concentration of MMF may be relatively high and consequently only slightly reduce the above-cited side effects.

There is therefore a need to significantly reduce the local concentration of MMF.

### SUMMARY OF THE INVENTION

It is the object of the following invention to address one or more of the aformentioned needs.

The minimum transit time in the small intestine of standard oral dosage forms is described in the scientific literature (Oral modified-release drug delivery systems, page 993, The Science and practice of pharmacy, Remington, Twenty-second edition) as being about 2 hours in the fasted state implying a maximum mean dissolution time of the dose of MMF in less than 120 minutes. In addition, the in vitro release rate of DMF (Tecfidera® 240 mg) is about 5% of the dose per minute for the first 15 minutes of dissolution. Halving the release rate, i.e. to about 2.5% of the dose per minute, should therefore generate a significant reduction in the local concentration of MMF. For these reasons, the invention preferably aims at releasing the MMF at an in-vitro release rate of below 2.5% of the dose per minute, with 100% of the dose being released in less than or equal to 120 minutes. A 90 minute mean dissolution time for the dose is considered an acceptable low limit value subject to a release rate below 2.5% of the dose per minute and taking into account the estimate of 30 minutes for the maximum dissolution time of an enteric hard capsule when the capsule reaches the small intestine. Indeed, the drug delivery system according to the invention may advantageously be designed to be added into an enteric hard capsule to avoid the release of the MMF in the stomach.

Polyethylene glycol (PEG)-based sustained release formulations are well described in scientific literature. According to the state of the art, the rate of release of water-soluble drugs decreases with the increasing molecular weight of the PEG. In other words, the PEG formulations with higher molecular weights should result in longer dissolution times. In contrast, the PEG-based sustained release formulations of the invention show unexpected results in meeting the target mean dissolution time for the lowest PEG molecular weights e.g. 4,000.

Based on the results of the experiments, the inventors have found that the PEG-based formulations with relatively high proportions of MMF, e.g. from 50% w/w to 60% w/w, do not follow the observations of the current state of the art. Indeed, the mean dissolution time is not impacted by the PEG molecular weight within these ranges of proportions i.e. 50% to 60% w/w of MMF and 50% to 40% of PEG respectively.

The inventors have also found that there is an optimal MMF proportion i.e. 60% w/w to reach a sustained release effect meeting the optimal maximum dissolution time (between 90 minutes to 120 minutes) whatever the molecular weight of the PEG e.g. from 4,000 to 35,000.

Surprisingly, the inventors have now found that when a PEG matrix comprises 50% w/w to 60% w/w of MMF, the release kinetics e.g. mean dissolution time, of the MMF from the PEG matrix can be slowed and an optimal sustained release can be achieved. Said decreased mean dissolution time and sustained release may result in lower amounts of MMF being released per time unit in the gastrointestinal tract following administration of such drug delivery system.

The inventors have also now found that when a PEG with a molecular weight of less than 35,000, and more surprisingly of low molecular weight e.g. less than 20,000 e.g. 4,000, comprises 50% w/w to 60% w/w of MMF, the release kinetics e.g. mean dissolution time, of said MMF from said PEG matrix can be slowed and an optimal sustained release can be achieved. Said decreased mean dissolution time and sustained release may result in lower amounts of MMF being released per time unit in the gastrointestinal tract following administration of such drug delivery system, and may thereby reduce side effects associated with the oral administration of DMF.

The invention therefore concerns a sustained release drug delivery system, also named dosage formulation, comprising a monolithic solid PEG matrix and MMF characterized by the following conditions:
the MMF being dispersed under solid state in the said matrix, and
the system comprising 50% to 60% w/w of MMF.

Preferably, the invention consists of an oral PEG-based drug delivery system releasing MMF over an extended period of time.

According to an aspect of the present invention, the typical in vitro release rate of the drug delivery system is less than 2.5% of the effective dose of MMF per minute and the mean dissolution time of 100% of the effective dose of MMF is equal to or less than 120 minutes (in the test conditions of the examples).

According to yet another aspect of the present invention, the MMF is dispersed under solid state in the monolithic solid PEG matrix.

According to yet another aspect of the invention, the monolithic solid PEG matrix comprises more than or equal to 50% w/w of MMF and a maximum of 50% w/w of PEG.

According to yet another aspect of the present invention, the PEG matrix comprises from about 55% w/w to about 60% w/w of MMF.

According to yet another aspect of the present invention, the PEG matrix comprises 60% w/w of the MMF.

According to yet another aspect of the present invention, the PEG matrix comprises one PEG or a combination of PEGs having a molecular weight equal to or less than 20,000 and at minimum a proportion of 50% w/w of the total matrix weight.

According to yet another aspect of the present invention, the matrix has a cylindrical shape.

Preferably, the cylindrical shape has a diameter from 4.8 mm to 6.2 mm and a height between 5 mm and 17 mm. Its surface-to-volume ratio is advantageously 1.1 or less.

It is yet another aspect of the present invention to provide a sustained release drug delivery system for use in the treatment of an autoimmune disease wherein the autoimmune disease is preferably psoriasis or multiple sclerosis.

It is yet another aspect of the present invention to provide a method of treating a disease in a subject wherein said method comprises the step of orally administering to said subject an effective amount of a drug, said disease being preferably selected from the group of autoimmune disease consisting of psoriasis or multiple sclerosis.

Possible embodiments of the invention are set out in the claims, in the detailed description and in the accompanying drawings included herein.

### DETAILED DESCRIPTION OF THE INVENTION

The invention will be better understood in the present chapter, with some non-limiting examples.

### BRIEF DESCRIPTION OF THE FIGURES

FIG. 1: Interaction plot for mean dissolution time (min) of differing concentrations of MMF dependent on the molecular weight of the PEG.

FIG. 2: Interaction plot for mean dissolution time (min) of differing molecular weights of PEG dependent on the MMF concentration.

FIG. 3: Main Effects Plot comparing the effects of the different PEG matrices and different concentrations of MMF on the dissolution time (min).

FIG. 4: Comparison of dissolution profiles in FaSSIF pH 6.5 of the invention/MMF and Tecfidera®/DMF.

FIG. 5: Comparison of dissolution profiles in phosphate buffer 6.8 of the invention containing PEG 4,000 or 20,000

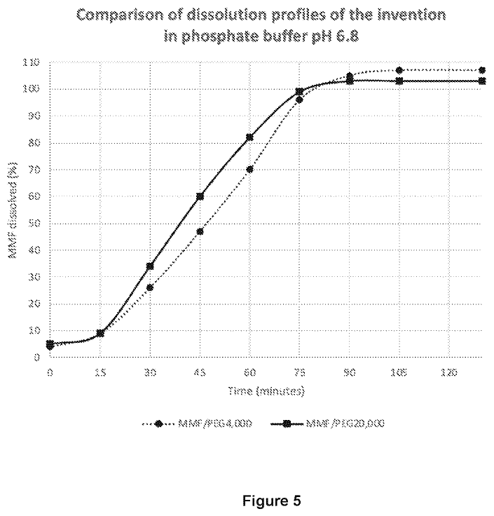

### DEFINITIONS

The term “active ingredient” as used herein refers to an ingredient that provides a therapeutic effect.

The term “effective dose” as used herein refers to a dose of an active ingredient sufficient to achieve a therapeutic effect in a subject in need thereof. For example, an effective dose of a drug useful for treating multiple sclerosis may be a dose capable of reducing the risk of relapse and/or the severity of relapses and/or the frequency of relapses, and/or of relieving one or more symptoms associated with the disease.

The term “sustained release” as used herein refers to the release of the MMF from the drug delivery system of the invention over an extended period of time in a continuous, discontinuous, linear or non-linear manner.

The term “extended period” as used herein refers to a continuous period of time of at least 1 hour and about 2 to 3 hours.

The term “drug delivery system” as used herein refers to a formulation that enables the introduction of an active ingredient into the body and improves its efficacy and/or safety by controlling the rate, time and place of release of drugs in the body.

The term “matrix” as used herein refers to a solid matrix obtained by melting the PEG and mixing the MMF (under solid state) in the liquified PEG. The mix is cooled down to produce a solid matrix. The matrix obtained is not a tablet as tablets are obtained by pressing a blend of solid powders between punches of a tableting machine. The “matrix” is a single excipient composition which is different by design to the composition of a tablet which requires at least a diluent, a disintegrant and a lubricant including excipients for modified release when required.

The surface-to-volume ratio of the drug delivery system defines its drug loading capacity and the relationship between the dose and the available contact surface when released in the gastrointestinal tract fluid. It may be expressed in mm² per mm³.

In a first example there is provided a sustained release drug delivery system comprising MMF dispersed under solid state in a monolithic solid PEG cylindrical shape matrix, wherein the monolithic solid PEG matrix comprises 50% w/w of MMF or more.

The minimum amount of PEG in such a monolithic solid cylindrical shape matrix may be 50% w/w.

The monolithic solid PEG cylindrical shape matrix may comprise from about 50% w/w to about 60% w/w of MMF.

In an embodiment of the invention the average molecular weight of said PEG is less than 35,000.

The average molecular weight of said PEG may be equal to or less than 20,000 or less than 8,000 or 4,000.

MMF is the active metabolite of DMF. Furthermore, it is assumed that MMF may be more bioavailable than DMF due to the pre-systemic metabolism of DMF. Accordingly, in some embodiments of the invention, administering MMF instead of DMF may be beneficial because the increased bioavailability of MMF over DMF could mean that a lower dosage of the active metabolite (i.e. MMF) may be needed. This could offer the advantage of reducing side effects associated with the oral administration of DMF, because less active ingredient is required to be released, e.g. in the gastrointestinal tract.

The MMF of the invention may be in the form of milled or micronized, particles. In an example, the MMF is in the form of milled or micronized particles having a mean particle size distribution of from 0.5 μm to 1000 μm e.g. from 0.5 μm to 500 μm e.g. from 0.5 μm to 100 μm.

Particles with a mean particle size distribution of from 1 μm to 100 μm may be particularly appropriate.

In another example, the MMF is in the form of micronized particles having a mean particle distribution of from 1 μm to 10 μm.

In addition to the PEG the drug delivery system may comprise other excipients such as glidants and/or lubricants.

The excipients may comprise talc, magnesium stearate or compounds having similar lubricating properties.

The drug delivery system may comprise a monolithic solid cylindrical shape matrix having a surface-to-volume ratio of less than or equal to 1.1 and having a diameter from 4.8 mm to 6.2 mm and a length between 5 mm and 17 mm.

An additional advantage of the drug delivery system of the invention is that the MMF release profile may be modified by adjusting the amount of MMF loaded into the monolithic solid PEG matrix e.g. depending on whether a quicker or slower release is required.

In another example, the drug delivery system has a release profile that reduces high local concentrations of the active metabolite (i.e. MMF) in the gastrointestinal tract.

In another example the drug delivery system has a release profile that reduces side effects associated with high local concentrations of the active metabolite (i.e. MMF) in the gastrointestinal tract.

In another example the drug delivery system is comprised in a hard capsule.

In a more specific example, the drug delivery system is comprised in a hard capsule e.g. a hard capsule made from gelatine or cellulose.

The hard capsule comprising the drug delivery system may be an enteric hard capsule. The enteric hard capsule serves to delay the release of the drug delivery system until it reaches the gastrointestinal tract, thereby avoiding the release of said system in the stomach.

Following the dissolution of the enteric hard capsule, the MMF comprised therein may then be sustainably released as the matrix makes its way through the gastrointestinal tract and more specifically through the small intestine. In another example the drug delivery system is comprised in an enteric hard capsule e.g. an enteric hard capsule made from cellulosic derivates.

In addition to the MMF the drug delivery system may comprise other active ingredients e.g. teriflunomide, fingolimod, acetylsalicylic acid.

The drug delivery system of the invention may be used as a medicine. Accordingly, in another aspect of the invention there is provided a drug delivery system of the invention for use as a medicine.

Fumaric acid esters such as MMF are known to be potentially effective in the treatment of hyperproliferative, inflammatory, neurodegenerative, and autoimmune disorders e.g. acute dermatitis, adrenal leukodystrophy, age-induced genome damage, Alexander's disease, alopecia areata (totalis and universalis), Alper's disease, Alzheimer's disease, amyotrophic lateral sclerosis, angina pectoris, arthritis, asthma, autoimmune diseases, balo concentric sclerosis, Behcet's syndrome, bullous pemphigoid, Canavan disease, cardiac insufficiency including left ventricular insufficiency, central nervous system vasculitis, Charcot-Marie-Tooth disease, childhood ataxia with central nervous system hypomyelination, chronic active (lupoid) hepatitis, chronic dermatitis, chronic idiopathic peripheral neuropathy, chronic obstructive pulmonary disease, contact dermatitis, Crohn's disease and cutaneous Crohn's disease, cutaneous lupus, cutaneous sarcoidosis, diabetic retinopathy, fibromyalgia, graft versus host disease, granuloma annulare, granulomas including annulare, Grave's disease, Hashimoto's thyroiditis, hepatitis C viral infection, herpes simplex viral infection, human immunodeficiency viral infection, Huntington's disease, inflammatory bowel disease, irritable bowel disorder, ischemia, juvenile-onset diabetes mellitus, Krabbe disease, lichen planus, macular degeneration, mitochondrial encephalomyopathy, monomelic amyotrophy, multiple sclerosis, myocardial infarction, necrobiosis lipoidica, neurodegeneration with brain iron accumulation, neurodermatitis, neuromyelitis optica, neuropathic pain, neurosarcoidosis, F-κB mediated diseases, optic neuritis, organ transplantation rejection, paraneoplastic syndromes, Parkinson's disease, Pelizaeus-Merzbacher disease, pemphigus, pernicious anemia, primary lateral sclerosis, progressive supranuclear palsy, psoriasis, psoriatic arthritis, pyoderma gangrenosum, radiation induced dermatitis, radicular pain, radiculopathic pain, reperfusion injury, retinopathic pigmentosa, rheumatoid arthritis, sarcoidosis, sarcoidosis, Schilder's disease, sciatic pain, sciatica, Sjogren's syndrome, subacute necrotizing myelopathy, such as polyarthritis, Susac's syndrome, systemic lupus erythematosus, tumors, transverse myelitis, ulcerative colitis, and Zellweger syndrome.

In another example, the drug delivery system is for use in the treatment of autoimmune diseases such as psoriasis or multiple sclerosis.

The invention also provides a method of treating a disease in a subject wherein said method comprises the step of administering to said subject an effective amount of a drug delivered by the drug delivery system of the invention.

Particularly effective doses of MMF per day may be from 30 mg to 720 mg or from 90 mg to 500 mg.

In another example the effective amount of MMF within the drug delivery system is an amount equating to a dosage of MMF from 90 mg to 500 mg e.g. from 108 mg to 432 mg or e.g. from 216 mg to 432 mg per day.

### Example 1: Dissolution Times of MMF/PEG Matrices

A series of experiments has been performed according to the following factors and levels. In Tables 1 and 2 below, the “PEG Mw” means “PEG molecular weight”.

TABLE 1

| | Levels | | |
|---|---|---|---|
| | Low | Medium | High |
| PEG Mw | 4000 | 20000 | 35000 |
| MMF (%) | 40 | 60 | 80 |

The matrices are comprised of PEG and MMF. The method of preparation is the following:
Melt the PEG at 60° C.-70° C.
Add the MMF and mix until homogeneous state at 60° C.-70° C.
Add the mixture into the matrix mould (6 mm diameter)
After 10 minutes at room temperature, remove the matrix from the mould
Cut the matrix at the target length of 10 mm
The matrix is a monolithic solid of 6 mm diameter with a length of 10 mm (surface-to-volume ratio of 0.87)
The mean mass of the matrices is 343 mg.

The conditions of the dissolution test are as follows:
Medium: Fasted state simulated intestinal fluid (FaSSIF) pH 6.5
Volume: 200 ml
Temperature: 37° C.
Mixing apparatus: paddle
Mixing speed: 40 rpm

A preliminary test of solubility of the MMF in the FaSSIF medium has been performed in order to verify that the maximum amount of MMF used in the matrix can be dissolved in the volume of FaSSIF medium used. There is no bias identified regarding the maximum solubility of MMF.

All the results are shown in Table 2.

TABLE 2

| No | PEG Mw | MMF (% w/w) | Dissolution time (min) |
|---|---|---|---|
| 1 | 35000 | 80 | 87 |
| 2 | 35000 | 80 | 93 |
| 3 | 4000 | 40 | 66 |
| 4 | 35000 | 60 | 112 |
| 5 | 20000 | 80 | 86 |
| 6 | 20000 | 60 | 104 |
| 7 | 4000 | 40 | 58 |
| 8 | 4000 | 80 | 103 |
| 9 | 4000 | 80 | 109 |
| 10 | 20000 | 40 | 74 |
| 11 | 4000 | 60 | 102 |
| 12 | 35000 | 40 | 95 |
| 13 | 35000 | 40 | 92 |

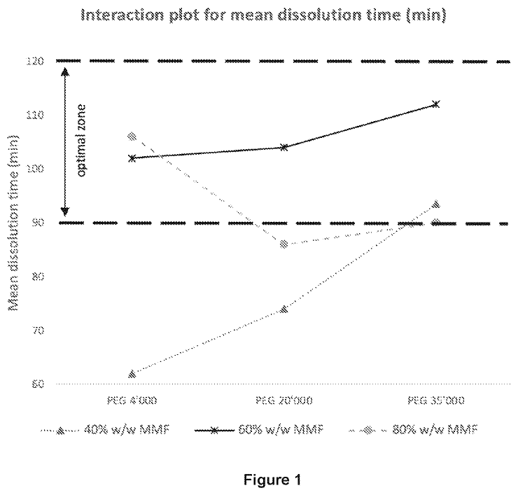

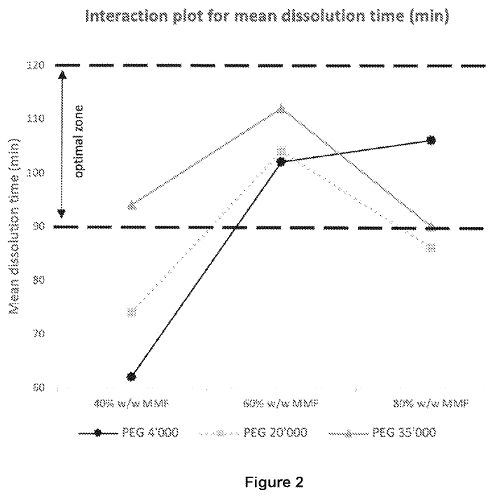

The interaction plots for the mean dissolution times shown in Table 2 are presented in FIGS. 1 and 2.

According to FIG. 1, the 40% w/w proportion of MMF in a PEG matrix follows the prior art. The higher the molecular weight of the PEG, the longer the dissolution time. The 60% w/w proportion of MMF presents a similar trend but the effect of the molecular weight of the PEG is about 3 times less pronounced (ranging from 102 minutes to 112 minutes, a range of 10 minutes) than the one obtained at 40% w/w (from 66 minutes to 95 minutes, a range of 29 minutes). In contrast, the 80% w/w of MMF reaches its longest mean dissolution time (103 minutes) with the lower molecular weight of PEG e.g. 4,000. Indeed, for higher molecular weights of PEG a faster dissolution time is observed (below or about the target of 90 minutes on average).

In FIG. 2, a threshold proportion of MMF is required for the lowest molecular weight of PEG e.g. 4,000 or 20,000 to reach the target dissolution time of 90 minutes. The required proportion is about 50% w/w of MMF as a threshold proportion. At 60% w/w proportion of MMF, the mean dissolution time is above the target of 90 minutes and below 120 minutes for all the molecular weights of PEG tested. At 80% w/w of MMF, a drop of mean dissolution time to about 90 minutes is observed for the highest molecular weights of PEG i.e. 20,000 and 35,000. Only the PEG with the lowest molecular weight i.e. 4,000 remains comparable to the 60% w/w proportion of MMF with the same molecular weight of PEG (106 minutes on average against 102 minutes).

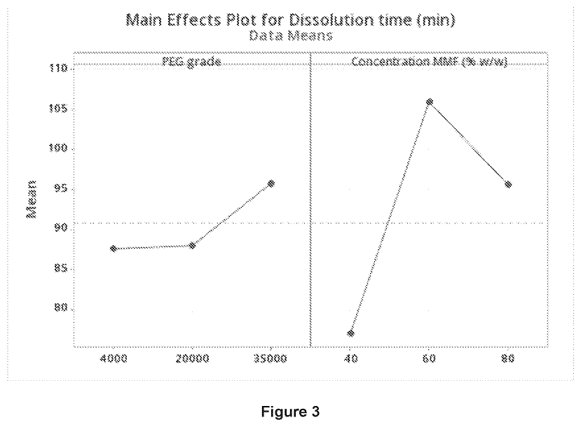

FIG. 3 compares the effects of the PEG matrices and different concentrations of MMF on the dissolution time. The effect of the proportion of MMF on the mean dissolution time is higher than that of the PEG molecular weight. Moreover, there is an optimal proportion of MMF i.e. between 50% w/w and 80% w/w and more accurately at 60% w/w to meet the optimal zone of dissolution time (between 90 minutes and 120 minutes). The effect of the proportion of MMF on the increase of the mean dissolution time presents a plateau at about 80% w/w.

There is an optimal proportion of MMF at 60% w/w to reach the target dissolution time window whatever the molecular weight of PEG used e.g. 4,000 e.g. 20,000 e.g. 35,000.

### Example 2: Comparison of Dissolution Profiles (Invention Vs Tecfidera®)

A drug delivery system according to the invention consists of 60% w/w MMF and 40% w/w PEG 4,000. The dose of MMF is 216 mg per matrix. The matrix is a single monolithic solid of approximately 6 mm diameter.

The commercial product used is Tecfidera® 120 mg. Two capsules are used to be equivalent to a dose of 240 mg of DMF. The contents of the capsules i.e. the enteric coated mini-tablets are poured directly into the medium.

The dissolution curves compare the percentage of MMF dissolved in the system according to the invention to the corresponding percentage of DMF (Tecfidera®) dissolved. 216 mg of MMF is equivalent to 240 mg of DMF in terms of the amount of the active metabolite.

The conditions of the dissolution test are as follows:
Medium: Fasted state simulated intestinal fluid (FaSSIF) pH 6.5
Volume: 300 ml
Temperature: 37° C.
Mixing apparatus: paddle
Mixing speed: 40 rpm

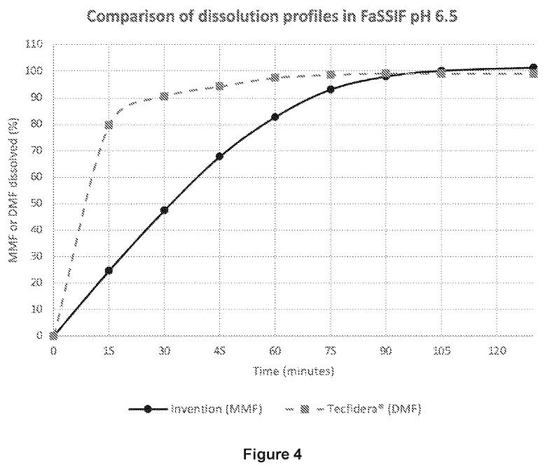

FIG. 4 compares the dissolution profiles of the system according to the present invention to those of DMF (Tecfidera®).

It is assumed that the release rate for the first 80% of MMF and DMF follows a linear model for both the system according to the invention and for Tecfidera®. The system of the invention allows a release rate of MMF of about 1.3% per minute whereas the release rate of Tecfidera® is about 5.3% DMF per minute.

The dissolution time of 100% of the dose of MMF is about 105 minutes thus meeting the target range of between 90 minutes and 120 minutes.

### Example 3: Comparison of Dissolution Profiles of the Invention (with Different PEG Mw) in Enteric Hard Capsule

A drug delivery system with a dose of 216 mg of MMF per matrix according to the invention consists of:
Matrix 1: 60% w/w MMF and 40% w/w PEG 4,000 in enteric hard capsule
Matrix 2: 60% w/w MMF and 40% w/w PEG 20,000 in enteric hard capsule

The conditions of the dissolution test are as follows:
Medium: phosphate buffer pH 6.8
Volume: 300 ml
Temperature: 37° C.
Mixing apparatus: paddle
Mixing speed: 40 rpm

It is assumed a linear release of the MMF from 15 minutes to 75 minutes for both matrices (R²=0.994). The average release rate of MMF is 1.5% per minute whatever the PEG molecular weight i.e. PEG 4,000 or PEG 20,000.

1. A sustained release drug delivery system comprising a monolithic solid polyethylene glycol (PEG) matrix and monomethyl fumarate (MMF) characterized by the following conditions:
the MMF being dispersed under solid state in the said matrix, and
the system comprising from 50% to 60% w/w of MMF.

2. System according to claim 1 wherein the PEG is a single type of PEG or a combination of PEGs having a molecular weight of 20,000 or less.

3. System according to claim 2 wherein the PEG molecular weight ranges from 4,000 to 20,000.

4. System according to claim 3 wherein the PEG molecular weight is 4,000.

5. System according to claim 1, wherein the PEG matrix comprises glidants and/or lubricants such as talc or magnesium stearate.

6. System according to claim 1, comprising several monolithic solid PEG matrices.

7. Hard capsule filled with the drug delivery system according to claim 1.

8. Capsule according to claim 7 wherein said capsule is an enteric hard capsule

9. System as defined in claim 1 for oral administration.

10. System as defined in claim 1 for use in the treatment of an autoimmune disease wherein the autoimmune disease is preferably psoriasis or multiple sclerosis.

11. System as defined in claim 1 for use in a daily administration of MMF in an amount of from 190 mg to 432 mg and preferably from 216 mg to 432 mg.

12. The system according to claim 1 wherein the matrix has a cylindrical shape.

13. The system according to claim 12 wherein the cylindrical shape has a diameter of between 4.8 mm and 6.2 mm and a height between 5 mm and 17 mm.
